SEQUENCE LISTING (64 sequences)

SEQ ID NO: 1 - DNA, 361 bp, Mus musculus
ggtgtccagtgtgaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtggtggtagttacacctactatccagacagtgtgaaggggcgattcaccatctccagagacaatgccaagaacaccctgtacctgcaaatgagcagtctgaagtctgaggacacagccatgtattactgtgcaagacggattactacggtagtagcccggtcgttctactttgactactggggccaaggca

SEQ ID NO: 2 - protein, 120 aa, Mus musculus
GVQCEVQLVESGGDLVKPGGSLKLSCAASGFTFSSYGMSWVRQTPDKRLEWVATISSGGSYTYYPDSVKGRFTISRDNAKNTLYLQMSSLKSEDTAMYYCARRITTVVARSFYFDYWGQG

SEQ ID NO: 3 - DNA, 102 bp, Mus musculus
ggtgtccagtgtgaggtgcagctggtggagtctgggggagacttagtgaagcctggagggtccctgaaactctcctgtgcagcctctggattcactttcagt

SEQ ID NO: 4 - protein, 34 aa, Mus musculus
GVQCEVQLVESGGDLVKPGGSLKLSCAASGFTFS

SEQ ID NO: 5 - DNA, 15 bp, Mus musculus
agctatggcatgtct

SEQ ID NO: 6 - protein, 5 aa, Mus musculus
SYGMS

SEQ ID NO: 7 - DNA, 42 bp, Mus musculus
tgggttcgccagactccagacaagaggctggagtgggtcgca

SEQ ID NO: 8 - protein, 14 aa, Mus musculus
WVRQTPDKRLEWVA

SEQ ID NO: 9 - DNA, 51 bp, Mus musculus
accattagtagtggtggtagttacacctactatccagacagtgtgaagggg

SEQ ID NO: 10 - protein, 17 aa, Mus musculus
TISSGGSYTYYPDSVKG

SEQ ID NO: 11 - DNA, 96 bp, Mus musculus
cgattcaccatctccagagacaatgccaagaacaccctgtacctgcaaatgagcagtctgaagtctgaggacacagccatgtattactgtgcaaga

SEQ ID NO: 12 - protein, 32 aa, Mus musculus
RFTISRDNAKNTLYLQMSSLKSEDTAMYYCAR

SEQ ID NO: 13 - DNA, 42 bp, Mus musculus
cggattactacggtagtagcccggtcgttctactttgactac

SEQ ID NO: 14 - protein, 14 aa, Mus musculus
RITTVVARSFYFDY

SEQ ID NO: 15 - DNA, 13 bp, Mus musculus
tggggccaaggca

SEQ ID NO: 16 - protein, 4 aa, Mus musculus
WGQG

SEQ ID NO: 17 - DNA, 327 bp, Mus musculus
ggttccactggtgacattgtgctgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggacca

SEQ ID NO: 18 - protein, 109 aa, Mus musculus
GSTGDIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGP

SEQ ID NO: 19 - DNA, 81 bp, Mus musculus
ggttccactggtgacattgtgctgacacagtctcctgcttccttagctgtatctctggggcagagggccaccatctcatac

SEQ ID NO: 20 - protein, 27 aa, Mus musculus
GSTGDIVLTQSPASLAVSLGQRATISY

SEQ ID NO: 21 - DNA, 45 bp, Mus musculus
agggccagcaaaagtgtcagtacatctggctatagttatatgcac

SEQ ID NO: 22 - protein, 15 aa, Mus musculus
RASKSVSTSGYSYMH

SEQ ID NO: 23 - DNA, 45 bp, Mus musculus
tggaaccaacagaaaccaggacagccacccagactcctcatctat

SEQ ID NO: 24 - protein, 15 aa, Mus musculus
WNQQKPGQPPRLLIY

SEQ ID NO: 25 - DNA, 21 bp, Mus musculus
cttgtatccaacctagaatct

SEQ ID NO: 26 - protein, 7 aa, Mus musculus
LVSNLES

SEQ ID NO: 27 - DNA, 96 bp, Mus musculus
ggggtccctgccaggttcagtggcagtgggtctgggacagacttcaccctcaacatccatcctgtggaggaggaggatgctgcaacctattactgt

SEQ ID NO: 28 - protein, 32 aa, Mus musculus
GVPARFSGSGSGTDFTLNIHPVEEEDAATYYC

SEQ ID NO: 29 - DNA, 27 bp, Mus musculus
cagcacattagggagcttacacgttcg

SEQ ID NO: 30 - protein, 9 aa, Mus musculus
QHIRELTRS

SEQ ID NO: 31 - DNA, 12 bp, Mus musculus
gaggggggacca

SEQ ID NO: 32 - protein, 4 aa, Mus musculus
EGGP

SEQ ID NO: 33 - DNA, 348 bp, Mus musculus
ggtgtccactctgaggtccagctgcaacagtctggacctgagctggtgaagcctggagcttcaatgaagatatcctgcaaggcttctggttactcattcactggctacaccatgaactgggtgaagcagagccatggaaagaaccttgagtggattggacttattaatccttacaatggtggtactagctacaaccagaagttcaagggcaaggccacattaactgtagacaagtcatccagcacagcctacatggagctcctcagtctgacatctgaggactctgcagtctattactgtgcaagaggagggaaatttgctacggggacctcttactggggccaaggg

SEQ ID NO: 34 - protein, 116 aa, Mus musculus
GVHSEVQLQQSGPELVKPGASMKISCKASGYSFTGYTMNWVKQSHGKNLEWIGLINPYNGGTSYNQKFKGKATLTVDKSSSTAYMELLSLTSEDSAVYYCARGGKFATGTSYWGQG

SEQ ID NO: 35 - DNA, 102 bp, Mus musculus
ggtgtccactctgaggtccagctgcaacagtctggacctgagctggtgaagcctggagcttcaatgaagatatcctgcaaggcttctggttactcattcact

SEQ ID NO: 36 - protein, 34 aa, Mus musculus
GVHSEVQLQQSGPELVKPGASMKISCKASGYSFT

SEQ ID NO: 37 - DNA, 15 bp, Mus musculus
ggctacaccatgaac

SEQ ID NO: 38 - protein, 5 aa, Mus musculus
GYTMN

SEQ ID NO: 39 - DNA, 42 bp, Mus musculus
tgggtgaagcagagccatggaaagaaccttgagtggattgga

SEQ ID NO: 40 - protein, 14 aa, Mus musculus
WVKQSHGKNLEWIG

SEQ ID NO: 41 - DNA, 51 bp, Mus musculus
cttattaatccttacaatggtggtactagctacaaccagaagttcaagggc

SEQ ID NO: 42 - protein, 17 aa, Mus musculus
LINPYNGGTSYNQKFKG

SEQ ID NO: 43 - DNA, 96 bp, Mus musculus
aaggccacattaactgtagacaagtcatccagcacagcctacatggagctcctcagtctgacatctgaggactctgcagtctattactgtgcaaga

SEQ ID NO: 44 - protein, 32 aa, Mus musculus
KATLTVDKSSSTAYMELLSLTSEDSAVYYCAR

SEQ ID NO: 45 - DNA, 30 bp, Mus musculus
ggagggaaatttgctacggggacctcttac

SEQ ID NO: 46 - protein, 10 aa, Mus musculus
GGKFATGTSY

SEQ ID NO: 47 - DNA, 12 bp, Mus musculus
tggggccaaggg

SEQ ID NO: 48 - protein, 4 aa, Mus musculus
WGQG

SEQ ID NO: 49 - DNA, 321 bp, Mus musculus
ggggccatttcccaggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggagga

SEQ ID NO: 50 - protein, 107 aa, Mus musculus
GAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGG

SEQ ID NO: 51 - DNA, 78 bp, Mus musculus
ggggccatttcccaggctgttgtgactcaggaatctgcactcaccacatcacctggtgaaacagtcacactcacttgt

SEQ ID NO: 52 - protein, 26 aa, Mus musculus
GAISQAVVTQESALTTSPGETVTLTC

SEQ ID NO: 53 - DNA, 42 bp, Mus musculus
cgctcaagtactggggctgttacaactagtaactatgccaac

SEQ ID NO: 54 - protein, 14 aa, Mus musculus
RSSTGAVTTSNYAN

SEQ ID NO: 55 - DNA, 45 bp, Mus musculus
tgggtccaagaaaaaccagatcatttattcactggtctaataggt

SEQ ID NO: 56 - protein, 15 aa, Mus musculus
WVQEKPDHLFTGLIG

SEQ ID NO: 57 - DNA, 21 bp, Mus musculus
ggtaccaacaaccgagctcca

SEQ ID NO: 58 - protein, 7 aa, Mus musculus
GTNNRAP

SEQ ID NO: 59 - DNA, 96 bp, Mus musculus
ggtgttcctgccagattctcaggctccctgattggagacaaggctgccctcaccatcacaggggcacagactgaggatgaggcaatatatttctgt

SEQ ID NO: 60 - protein, 32 aa, Mus musculus
GVPARFSGSLIGDKAALTITGAQTEDEAIYFC

SEQ ID NO: 61 - DNA, 27 bp, Mus musculus
gctctatggtacagcaaccattgggtg

SEQ ID NO: 62 - protein, 9 aa, Mus musculus
ALWYSNHWV

SEQ ID NO: 63 - DNA, 12 bp, Mus musculus
ttcggtggagga

SEQ ID NO: 64 - protein, 4 aa, Mus musculus
FGGG

(19) United States
(12) Patent Application Publication
(10) Pub. No.: US 2012/0122126 A1
(43) Pub. Date: May 17, 2012

(54) ANTI-PSK ANTIBODY

(75) Inventors: Hirotaka Hoshi, Tokyo (JP); Hikaru Saito, Tokyo (JP); Motoyuki Uchida, Tokyo (JP)

(73) Assignee: Kureha Corporation, Tokyo (JP)

(21) Appl. No.: 13/386,653

(22) PCT Filed: Jul. 23, 2010
(86) PCT No.: PCT/JP2010/062414

§ 371 Date: Jan. 23, 2012

(30) Foreign Application Priority Data
Jul. 24, 2009 (JP) 2009-173732

(51) Int. Cl. G01N 33/566; C07K 16/18

(52) U.S. Cl. 435/7.92; 530/387.1; 530/387.3; 530/387.5

(57) ABSTRACT

An antibody which recognizes PSK is provided.

The object of the present invention is solved by an antibody characterized by recognizing PSK and inhibiting anti-tumor activity of PSK. In particular, the inhibition of anti-tumor activity of PSK is an inhibition of cytotoxic activity or an inhibition of anti-TGF-β1 activity of PSK. A physiologically active PSK can, using the present invention, be detected high-accurately and quantitatively. For example, the present invention is useful for detecting and measuring the physiologically active PSK contained in a medicine or a food and drink, and for analyzing pharmacokinetics after administrating the physiologically active PSK.

### TECHNICAL FIELD

The present invention relates to an anti-PSK antibody, a method for analyzing PSK, and a kit for analyzing PSK. More specifically, the invention relates to an antibody which binds to PSK, a method for analyzing PSK based on, for example, a ELISA method or a surface plasmon resonance method (SPR method: Biacore method) using the antibody, and a kit for analyzing PSK containing the antibody.

### BACKGROUND ART

The protein-bound polysaccharides extracted from Coriolus versicolor (Fr.) Quel. exhibit an anti-tumor activity or the like, and an anti-tumor agent which contains these protein-bound polysaccharides as an effective component is described in, for example, Japanese Unexamined Patent Publication No. 60-45533 (Patent Reference 1). Among the protein-bound polysaccharides, PSK (registered trademark) [product name: “KRESTIN” (registered trademark)] derived from Coriolus versicolor (Fr.) Quel. is characterized in that it exhibits anti-tumor activity not only by intradermal administration or intravenous administration, but also by oral administration, and it is also clinically used as a preparation for oral administration.

PSK is a protein-bound polysaccharide comprising about 18 to 38% by weight of proteins, and it has a molecular weight of 5000 of more (measured by the gel filtration method), for example, a molecular weight of 5000 to 300000 (gel filtration method). Sugar residue of the major fraction is β-D-glucan, and the structure of the glucan is a branched structure having 1→3, 1→4 and 1→6 bond.

PSK has been used as an anti-tumor agent as described above. It is reported that PSK has various physiological activities including an anti-tumor activity, a cytotoxic activity, an activity of inhibiting TGF-β1, an activity of inhibiting PDGF, and an activity of inducing cytokine production, or the like (Patent Reference 2). For product quality management of an anti-tumor agent containing PSK, direct measurement of the physiological activity of PSK is necessary to determine the level of physiological activity of PSK contained in the preparation. Thus, the process is cumbersome and time-consuming. Accordingly, it is desired to develop a method for simple measurement of the amount of physiologically active PSK.

Conventionally, as a method for detecting or measuring the amount of PSK, Limuls test, which is generally used for detecting LPS or β1, 3 glucan, is employed. However, in the Limulus test, polysaccharides other than PSK (such as laminarin and yeast glucan) having β1, 3 glucan structure, can be detected, and thus it is not a PSK-specific detection method. Therefore, the amount of physiologically active PSK cannot be specifically measured by the Limulus test. A method for detecting PSK based on the fluorescence antibody method using a rabbit polyclonal antibody against PSK is also reported (Non-Patent Reference 1). However, the fluorescence antibody method using a rabbit polyclonal antibody against PSK is also not a PSK-specific detection method. This is because, the antibody used in the method recognizes all of β1, 3 glucan structure, β1, 4 glucan structure, and β1, 6 glucan structure and detect any polysaccharides having those glucan structures. Further, since the PSK having no physiological activity is also detected, it cannot be used for quality management and the like of an anti-tumor agent (preparation).

### CITATION LIST

### Patent Reference

[Patent Reference 1] Unexamined Patent Publication No. 60-45533
[Patent Reference 2] Unexamined Patent Publication No. 8-208704

### Non-Patent Reference

[Non-Patent Reference 1] International Journal of Immunopharmacology (Netherlands) 1988, vol. 10, p. 103-109

### SUMMARY OF INVENTION

### Problems to be Solved by the Invention

An object of the invention is to provide a means for simple and highly accurate detection or measurement of physiologically active PSK that is contained in a drug, a food or a drink. Further, provided by the invention is a means for simple and highly accurate detection or measurement of physiologically active PSK that is contained in blood or tissues of a human body after administration of PSK.

The present inventors have conducted intensive studies to develop a method of specifically detecting or measuring the amount of physiologically active PSK, and as a result, they found that, by obtaining and using a monoclonal antibody having an activity of suppressing the cytotoxic activity of PSK and TGF-β1 inhibitory activity of PSK, physiologically active PSK can be conveniently detected or measured. Specifically, the monoclonal antibody used is a monoclonal antibody which binds to the physiologically active site showing the cytotoxic activity of PSK or the TGF-β1 inhibitory activity of PSK or to an epitope near the site. The physiologically active site is detected by the binding of the antibody, and thus it becomes possible to easily measure the amount of PSK having a physiologically active site by using the antibody.

The invention is based on the above findings.

### Means for Solving the Problems

The invention relates to an antibody characterized by recognizing PSK and inhibiting the antitumor activity of PSK.

According to the antibody of one preferable embodiment of the invention, the inhibition of the antitumor activity is due to inhibition of the cytotoxic activity of PSK.

According to the antibody of another preferable embodiment of the invention, the inhibition of the antitumor activity is due to suppression of the TGF-β1 inhibitory activity.

The antibody of the preferable embodiment of the invention has: (1) a heavy chain variable region domain comprising: the polypeptide of heavy chain complementarity determining region 1 consisting of an amino acid sequence of SEQ ID NO: 6, the polypeptide of heavy chain complementarity determining region 2 consisting of an amino acid sequence of SEQ ID NO: 10, and the polypeptide of heavy chain complementarity determining region 3 consisting of an amino acid sequence of SEQ ID NO: 14, and a light chain variable region domain comprising: the polypeptide of light chain complementarity determining region 1 consisting of an amino acid sequence of SEQ ID NO: 22, the polypeptide of light chain complementarity determining region 2 consisting of an amino acid sequence of SEQ ID NO: 26, and the polypeptide of light chain complementarity determining region 3 consisting of an amino acid sequence of SEQ ID NO: 30, or (2) a heavy chain variable region domain comprising: the polypeptide of the heavy chain complementarity determining region 1, the polypeptide of the heavy chain complementarity determining region 2, and the polypeptide of the heavy chain complementarity determining region 3, and a light chain variable region domain comprising: the polypeptide of the light chain complementarity determining region 1, the polypeptide of the light chain complementarity determining region 2, and the polypeptide of the light chain complementarity determining region 3, wherein each polypeptide region consists of an amino acid sequence with one or more amino acid deletions, substitutions, insertions, or additions in at least one of the following amino acid sequences: the amino acid sequence of SEQ ID NO: 6, the amino acid sequence of SEQ ID NO: 10, the amino acid sequence of SEQ ID NO: 14, the amino acid sequence of SEQ ID NO: 22, the amino acid sequence of SEQ ID NO: 26, and the amino acid sequence of SEQ ID NO: 30. Thus, according to the embodiment (2) above, one or more amino acids in the amino acid sequences which are comprised in the antibody of the embodiment (1) are deleted, substituted, inserted, or added.

The antibody of another preferable embodiment of the invention has: (1) a heavy chain variable region domain comprising: the polypeptide of heavy chain complementarity determining region 1 consisting of an amino acid sequence of SEQ ID NO: 38, the polypeptide of heavy chain complementarity determining region 2 consisting of an amino acid sequence of SEQ ID NO: 42, and the polypeptide of heavy chain complementarity determining region 3 consisting of an amino acid sequence of SEQ ID NO: 46, and a light chain variable region domain comprising: the polypeptide of light chain complementarity determining region 1 consisting of an amino acid sequence of SEQ ID NO: 54, the polypeptide of light chain complementarity determining region 2 consisting of an amino acid sequence of SEQ ID NO: 58, and the polypeptide of light chain complementarity determining region 3 consisting of an amino acid sequence of SEQ ID NO: 62, or (2) a heavy chain variable region domain comprising: the polypeptide of the heavy chain complementarity determining region 1, the polypeptide of the heavy chain complementarity determining region 2, and the polypeptide of the heavy chain complementarity determining region 3, and a light chain variable region domain comprising: the polypeptide of the light chain complementarity determining region 1, the polypeptide of the light chain complementarity determining region 2, and the polypeptide of the light chain complementarity determining region 3, wherein each polypeptide region consists of an amino acid sequence with one or more amino acid deletions, substitutions, insertions, or additions in at least one of the following amino acid sequence s: the amino acid sequence of SEQ ID NO: 38, the amino acid sequence of SEQ ID NO: 42, the amino acid sequence of SEQ ID NO: 46, the amino acid sequence of SEQ ID NO: 54, the amino acid sequence of SEQ ID NO: 58, and the amino acid sequence of SEQ ID NO: 62. Thus, according to the embodiment (2) above, one or more amino acids in the amino acid sequences which are comprised in the antibody of the embodiment (1) are deleted, substituted, inserted, or added.

According to the antibody of a preferable embodiment of the invention, it competes with the antibody above for binding to an epitope. Further, according to the antibody of a preferable embodiment of the invention, it binds to the epitope to which the antibody binds. Still further, according to a preferable embodiment, the antibody is an IgM antibody.

According to a preferable embodiment of the invention, the antibody is a chimeric antibody, a CDR-grafted antibody, or a human type antibody. In particular, the chimeric antibody is preferably a chimeric antibody with a human antibody and the CDR-grafted antibody is a CDR-grafted antibody with a human antibody. In addition to those, the chimeric antibody is preferably a chimeric antibody with IgW, IgNAR, IgX, or IgY and the CDR-grafted antibody is preferably a CDR-grafted antibody with IgW, IgNAR, IgX, or IgY.

Further, the invention relates to an antigen binding fragment selected from a group consisting of Fab, Fab′, F(ab′)₂, Fv fragment, a diabody, a single chain antibody molecule, and a multi-specific antibody, of the above antibody.

Further, the invention relates to a method for analyzing PSK using the antibody or antigen binding fragment thereof.

Further, the invention relates to a kit for analysis of PSK comprising the antibody or antigen binding fragment thereof.

Further, the invention relates to use of the antibody or antigen binding fragment thereof, for analysis of PSK.

Still further, the invention relates to use of the antibody or antigen binding fragment thereof, for manufacture of a kit for analysis of PSK.

### Advantageous Effects of Invention

According to the present invention, physiologically active PSK can be detected with high accuracy and quantitative performance. The invention is useful for the detection and measurement of physiologically active PSK that is contained in a drug, a food or a drink, and for the understanding in vivo kinetics after administration of physiologically active PSK.

Further, as the antibody of the present invention can inhibit the cytotoxic activity of PSK and TGF-β1 inhibitory activity of PSK, it is believed that it binds to a physiologically active site of PSK or a region near thereto. As such, it can be used for a study of identifying active sites for cytotoxic activity of PSK and TGF-β1 inhibitory activity of PSK.

### BRIEF DESCRIPTION OF DRAWINGS

FIG. 1 illustrates a result of antibody titre of Balb/c mouse immunized with PSK measured by ELISA, wherein the horizontal axis of the graph indicates serum dilution ratio and the vertical axis of the graph indicates absorbance (titre).

FIG. 2 illustrates antibody titre of 2G9 and 5G5 antibodies that are purified from the mouse ascites, wherein the horizontal axis of the graph indicates antibody concentration and the vertical axis of the graph indicates absorbance (titre).

FIG. 3 illustrates the reactivity of 2G9 antibody and 5G5 antibody examined by polysaccharide competition test.

FIG. 4 illustrates the reactivity of 2G9 antibody and 5G5 antibody examined by competition test using PSK in which the proteins are hydrolyzed with hydrazine.

FIG. 5 illustrates the result of a competition test between 2G9 antibody and 5G5 antibody for binding to PSK.

FIG. 6-a illustrates the nucleotide sequence and amino acid sequence of the heavy chain variable region domain of 2G9 antibody.

FIG. 6-b illustrates the nucleotide sequence and amino acid sequence of the light chain variable region domain of 2G9 antibody.

FIG. 6-c illustrates the nucleotide sequence and amino acid sequence of the heavy chain variable region domain of 5G5 antibody.

FIG. 6-d illustrates the nucleotide sequence and amino acid sequence of the light chain variable region domain of 5G5 antibody.

FIG. 7 illustrates the neutralizing activity of 2G9 antibody against cytotoxic activity of PSK.

FIG. 8 illustrates a photographic image obtained by immunohistochemical staining of a MethA tumor using 2G9 antibody, wherein the tumor has been implanted in a mouse (top: x 400, bottom: x 900).

FIG. 9 illustrates the function of 2G9 antibody and 5G5 antibody for suppressing the TGF-β1 inhibitory activity of PSK.

### DESCRIPTION OF EMBODIMENTS

### Embodiment 1

Before explaining one embodiment of the anti-PSK antibody relating to the present invention, a general explanation of an antibody is given herein below to aid understanding of the present invention.

Antibody is also referred to as immunoglobulin, and the basic structural unit of an antibody is known as a tetramer. Each tetramer consists of two identical pairs of polypeptides, and each pair consists of a light chain (L chain) of about 25 kD and a heavy (H chain) of about 50 to 70 kD. The light chain is classified into either kappa chain and lambda chain. Meanwhile, the heavy chain is classified into any one of gamma chain, mu chain, alpha chain, delta chain, and epsilon chain, and according to the type of each heavy chain, an antibody is classified into isotypes of IgG, IgM, IgA, IgD, and IgE.

The amino terminal of a heavy chain and a light chain is a polypeptide variable region which consists of about 100 to 110 or more amino acids and mainly contributes to antigen recognition. The carboxy terminal of each chain is a polypeptide constant region that mainly contributes to an effector function. Within the light chain and heavy chain, the variable region and the constant region are joined via a “J” region of 12 or more amino acids. The heavy chain also contains a “D” region with 10 or more amino acids. Further, the variable region at the amino terminal of the light chain and heavy chain form an antibody binding site, and therefore an intact antibody has two antigen-binding sites.

Specifically, the heavy chain has, from the amino terminal thereof, a polypeptide variable region (hereinafter referred to as heavy chain variable region domain (VH)) and polypeptides of three domains in a constant region: heavy chain constant region domain 1 (CH 1), heavy chain constant region domain 2 (CH 2), and heavy chain constant region domain 3 (CH 3) in that order. The heavy chain variable region domain contains three complementarity determining regions: heavy chain complementarity determining region 1 (hereinafter referred to as H-CDR1), heavy chain complementarity determining region 2 (hereinafter referred to as H-CDR2), and heavy chain complementarity determining region 3 (hereinafter referred to as H-CDR3), and the three complementarity determining regions are surrounded by a framework of the heavy chain variable region. Specifically, the framework of the heavy chain variable region consists of four polypeptides in the framework region: from the amino terminal, H-FR1, H-FR2, H-FR3 and H-FR1. Thus, the heavy chain variable region domain contains H-FR1, H-CDR1, H-FR2, H-CDR2, H-FR3, H-CDR3, and H-FR4 in the order.

Meanwhile, the light chain has, from the amino terminal thereof, a polypeptide variable region (hereinafter referred to as light chain variable region domain (VL)) and a polypeptide in a constant region (hereinafter referred to as light chain constant region domain (CL)) in the order. The light chain variable region domain contains three complementarity determining regions: light chain complementarity determining region 1 (hereinafter referred to as L-CDR1), light chain complementarity determining region 2 (hereinafter referred to as L-CDR2), and light chain complementarity determining region 3 (hereinafter referred to as L-CDR3), and the three complementarity determining regions are surrounded by a framework of the light chain variable region. Specifically, the framework of the light chain variable region consists of four polypeptides in the framework region: from the amino terminal, L-FR1, L-FR2, L-FR3, and L-FR1. Thus, the light chain variable region domain contains polypeptides of each of L-FR1, L-CDR1, L-FR2, L-CDR2, L-FR3, L-CDR3, and L-FR4 in that order.

In connection with this, assignment of the polypeptide consisting of an amino acid sequence which constitutes each domain of the polypeptide in the heavy chain and light chain variable region is based on the rules described by Kabat (1991) and/or Chothia and Lesk, J. Mol. Biol. 196: 901-917 (1987), and; Chothia, et. Al., Nature 342: 878-883 (1989).

Further, the amino acid sequence of the polypeptide in the heavy chain variable region domain and light chain variable region domain of the anti-PSK antibody of the present invention is not limited as long as the antigen binding site consisting of the heavy chain variable region domain and light chain variable region domain binds to PSK, the epitope bound with the antigen binding site is a PSK specific epitope, and the cytotoxic activity of PSK is suppressed by the binding of the antibody.

As used herein, the term “antibody” further includes a chimeric antibody, a CDR-grafted antibody, and a human type antibody. Thus, unless specifically described otherwise, the term expressed as “antibody” means any of these antibodies.

The chimeric antibody can be produced by, for example, ligating a DNA encoding the heavy chain variable region domain and light chain variable region domain of a mouse to a DNA encoding the polypeptide of a constant region of another kind of antibody, such as a human antibody, incorporating it into an expression vector, and introducing the vector to a host. The source of the polypeptides of the heavy chain variable region domain, light chain variable region domain, and constant regions that are used for a chimeric antibody is not specifically limited, and an immunoglobulin isotype in any of mammals, amphibians, birds, cartilaginous fishes, and teleost fishes can be used. For example, by using polypeptides of the heavy chain variable region domain and light chain variable region domain of mouse IgM and the constant region of human IgM or IgG, a chimeric antibody can be obtained.

CDR-grafted antibody is obtained by replacing a complementarity determining region (CDR) of a mouse antibody with a complementarity determining region of another kind of an antibody, such as a human antibody. Specifically, a DNA sequence designed to link CDR of a mouse antibody to the framework region (FR) of a human antibody is synthesized by PCR by using several oligonucleotides that are prepared to have overlapping parts at each of their terminals. Then, by linking the resulting DNA to a DNA which encodes the C region of a human antibody, incorporating the resultant to an expression vector, and introducing the vector to a host, a CDR-grafted antibody can be obtained. The source of the polypeptide of the complementarity determining region, framework region, and constant region used for CDR-grafted antibody is not specifically limited, and an immunoglobulin isotype in any of mammals, amphibians, birds, cartilaginous fishes, and teleost fishes can be used. For example, a CDR-grafted antibody can be obtained by using polypeptides of the complementarity determining region of mouse IgM and the framework region and the constant region of human IgM or IgG. Further, an antigen binding fragment of a CDR-grafted antibody can be obtained by using a complementarity determining region of a mouse and a framework region of human IgM and IgG.

Further, as used herein, the term “human type antibody” means an antibody obtained from a transgenic animal to which a gene of human antibody is introduced or a monoclonal antibody which can be obtained by cell fusion between a cell producing a human antibody and a myeloma cell.

### [1] Anti-PSK Antibody of the Invention

### (Outline of Anti-PSK Antibody)

Herein below, one preferable embodiment of the anti-PSK antibody of the present invention is explained as an embodiment 1. The anti-PSK antibody recognizes PSK. PSK can be obtained by extracting cell bodies of Coriolus versicolor (Fr.) Quel. Strain CM101 [FERM-P2412 (ATCC20547)] with an aqueous solution like hot water and alkali solution (for example, hydroxides of alkali metals, in particular, an aqueous solution of sodium hydroxide) followed by purification and drying. Sugar residues of major fractions are β-D-glucan, and the structure of glucan is a branched structure containing β1→3, β1→4 and β1→6 bond. The major constituting monosaccharide is glucose or mannose, and it contains about 18 to 38% by weight of protein. The protein-constituting amino acids are mostly an acidic amino acid such as asparaginic acid or glutamic acid and a neutral amino acid such as valine or leucine, while only a few basic amino acids such as lysine and arginine are contained. The anti-PSK antibody is soluble in water but hardly soluble in methanol, pyridine, chloroform, benzene, or hexane.

The anti-PSK antibody does not bind to laminarin, yeast glucan, or dextran. Laminarin is a storage polysaccharide found in kelp, and it is a water soluble glucan with relatively low molecular weight, having β-1, 3 bond and β1, 6 bond glucose as a main skeleton. Yeast glucan is a glucan present in yeast cell membranes, and it mostly contains β-1, 3 glucan and a little amount of β1, 6 glucan. Dextran is a polysaccharide consisting only of a glucose that is produced by lactic acid bacteria using sucrose as a starting material, and it contains a relatively large amount of α1, 6 glucan. The anti-PSK antibody does not recognize either laminarin or yeast glucan, and therefore it cannot recognize β1, 3 glucan and β1, 6 glucan. Further, as it does not recognize dextran, it cannot recognize α1, 6 glucan. Thus, the epitope to which the anti-PSK antibody binds has a structure present on β1, 3 glucan, β1, 4 glucan, or β1, 6 glucan of PSK.

The anti-PSK antibody can also recognize protein-hydrolyzed PSK wherein the protein residues of PSK are hydrolyzed by hydrazine. Thus, the epitope to which the anti-PSK antibody binds is an epitope that is not affected by hydrazine treatment of PSK.

### (Activities of Anti-PSK Antibody)

As it has an anti-tumor activity, PSK can be used as an anti-tumor agent for chemotherapy for treating a tumor.

Included in the anti-tumor activity of PSK is “cytotoxic activity”, “TGF-β1 inhibitory activity”, “PDGF inhibitory activity”, and “cytokine production inducing activity”, and based on at least one or a combination of two or more of these, the anti-tumor activity of PSK is exhibited.

As a major physiological activity exhibiting an anti-tumor effect, PSK to which the anti-PSK antibody binds has a cytotoxic activity. The anti-PSK antibody can suppress the cytotoxic activity. The cytotoxic activity of PSK is an activity of directly damaging and killing tumor cells when tumor cells are cultured in vitro with PSK. Suppression of the cytotoxic activity by an anti-PSK antibody can be confirmed by improved survival ratio of the tumor cells when the anti-PSK antibody is added to culture of tumor cells and PSK. The improved survival ratio means suppression of the cytotoxic activity, even if it is only a minor improvement. Specifically, the suppression of the cytotoxic activity of PSK by anti-PSK antibody can be measured as follows.

When a certain number of PSK-sensitive tumor cells (e.g., colon cancer cell line Colon 26) and PSK at a certain concentration (e.g., 10 μg/mL, 100 μg/mL, or the like) are cultured in vitro, the tumor cells are damaged and destroyed within three days. However, by adding the anti-PSK antibody at a certain concentration (e.g., 10 μg/mL, 100 μg/mL, or the like) to a culture containing Colon 26 and PSK, the cytotoxic activity of PSK is suppressed so that survival ratio of the tumor cells is improved.

For example, when Colon 26 cells are cultured with PSK at a concentration of 100 μg/mL as shown in the Examples described below, the survival ratio of Colon 26 cells is about 10% after three days. However, by adding the anti-PSK antibody at a concentration of 100 μg/mL, the survival ratio of Colon 26 cells is recovered to 80%.

The anti-PSK antibody can also suppress the “TGF-β1 inhibitory activity”. The TGF-β1 inhibitory activity by PSK is to recover in vitro proliferation of TGF-β1 sensitive cells based on PSK's neutralization of TGF-β1 function against inhibited proliferation of the sensitive cells. Meanwhile, the suppression of the TGF-β1 inhibitory activity by anti-PSK antibody can be identified by suppressed cell proliferation after adding the anti-PSK antibody to the cell culture described above. Even a small inhibition ratio on cell proliferation means that the antibody has a TGF-β1 inhibitory activity. Specifically, the anti-PSK antibody's activity of suppressing the TGF-β1 inhibitory activity of PSK can be measured as follows.

When a certain number of TGF-β1-sensitive cells (e.g., Mv1 Lu cells) and TGF-β1 at a certain concentration (e.g., 1 ng/mL) are cultured, proliferation of the TGF-β1-sensitive cells is suppressed. By adding PSK at a certain concentration (e.g., 50 μg/mL) to the culture, proliferation of the TGF-β1-sensitive cells is recovered. Further, by adding an anti-PSK antibody at a certain concentration (e.g., 50 μg/mL) to the culture containing TGF-β1-sensitive cells, TGF-β1, and PSK, the TGF-β1 inhibitory activity of PSK is suppressed so that proliferation of TGF-β1-sensitive cells is suppressed.

For example, when Mv1Lu cells are cultured with TGF-β1 at a concentration of 1 ng/mL and PSK at a concentration of 50 μg/mL as shown in the Examples described below, survival ratio of Mv1Lu cells is about 80% after three days. However, by adding the anti-PSK antibody (2G9 antibody or 5G5 antibody) at a concentration of 50 μg/mL, the survival ratio of Mv1Lu cells is suppressed to about 50%.

### (Structure of Anti-PSK Antibody)

Hereinafter, the structure of the anti-PSK antibody is explained.

### Embodiment (A)

As a first embodiment of an anti-PSK antibody (hereinafter referred to as the embodiment (A)), an antibody having the following heavy chain variable region domain and light chain variable region domain may be described. The antibody of the embodiment (A) is represented by the 2G9 antibody described in the Examples. The heavy chain variable region domain preferably comprises the H-CDR1 consisting of an amino acid sequence (SYGMS) of SEQ ID NO: 6, the H-CDR2 consisting of an amino acid sequence (TISSGGSYTYYPDSVKG) of SEQ ID NO: 10, and the H-CDR3 consisting of an amino acid sequence (RITTVVARSFYFDY) of SEQ ID NO: 14. Further, the light chain variable region domain preferably comprises the L-CDR1 consisting of an amino acid sequence (RASKSVSTSGYSYMH) of SEQ ID NO: 22, the L-CDR2 consisting of an amino acid sequence (LVSNLES) of SEQ ID NO: 26, and the L-CDR3 consisting of an amino acid sequence (QHIRELTRS) of SEQ ID NO: 30.

Further, the heavy chain variable region domain of the antibody of the embodiment (A) comprises the H-CDR1 consisting of an amino acid sequence of SEQ ID NO: 6, the H-CDR2 consisting of an amino acid sequence of SEQ ID NO: 10, the H-CDR3 consisting of an amino acid sequence of SEQ ID NO: 14, and a framework of the heavy chain variable region domain. Most preferably, it is the heavy chain variable region domain consisting of an amino acid sequence of SEQ ID NO: 2. Further, the light chain variable region domain of the antibody comprises the L-CDR1 consisting of an amino acid sequence of SEQ ID NO: 22, the L-CDR2 consisting of an amino acid sequence of SEQ ID NO: 26, the L-CDR3 consisting of an amino acid sequence of SEQ ID NO: 30, and a framework of the light chain variable region domain. Most preferably, it is the light chain variable region domain consisting of an amino acid sequence of SEQ ID NO: 18.

### Embodiment (B)

As a second embodiment of an anti-PSK antibody (hereinafter referred to as the embodiment (B)), an antibody having the following heavy chain variable region domain and light chain variable region domain may be described. The antibody of the embodiment (B) is represented by the 5G5 antibody described in the examples below. The heavy chain variable region domain preferably comprises the H-CDR1 consisting of an amino acid sequence (GYTMN)) of SEQ ID NO: 38, the H-CDR2 consisting of an amino acid sequence (LINPYNGGTSYNQKFKG) of SEQ ID NO: 42, and the H-CDR3 consisting of an amino acid sequence (GGKFATGTSY) of SEQ ID NO: 46. Further, the light chain variable region domain preferably comprises the L-CDR1 consisting of an amino acid sequence (RSSTGAVTTSNYAN) of SEQ ID NO: 54, the L-CDR2 consisting of an amino acid sequence (GTNNRAP) of SEQ ID NO: 58, and the L-CDR3 consisting of an amino acid sequence (ALWYSNHWV) of SEQ ID NO: 62.

Further, the heavy chain variable region domain of the antibody of the embodiment (B) comprises the H-CDR1 consisting of an amino acid sequence of SEQ ID NO: 38, the H-CDR2 consisting of an amino acid sequence of SEQ ID NO: 42, the H-CDR3 consisting of an amino acid sequence of SEQ ID NO: 46, and a framework of the heavy chain variable region domain. Most preferably, it is the heavy chain variable region domain consisting of an amino acid sequence of SEQ ID NO: 34. Further, the light chain variable region domain of the antibody comprises the L-CDR1 consisting of an amino acid sequence of SEQ ID NO: 54, the L-CDR2 consisting of an amino acid sequence of SEQ ID NO: 58, the L-CDR3 consisting of an amino acid sequence of SEQ ID NO: 62, and a framework of the light chain variable region domain. Most preferably, it is the light chain variable region domain consisting of an amino acid sequence of SEQ ID NO: 50.

The H-CRD1, the H-CRD2, the H-CRD3, the L-CRD1, the L-CRD2, and the L-CRD3 of the anti-PSK antibody of the embodiment (A) and the embodiment (B) may have one or more deletions, substitutions, insertions, or additions of amino acids. The antigen binding site, which is formed with the heavy chain variable region domain and light chain variable region domain containing polypeptides with deletions, substitutions, insertions, or additions, binds to the same epitope to which 2G9 antibody or 5G5 binds. The cytotoxic activity of PSK can be suppressed by binding of the antibody.

Further, the polypeptide of the heavy chain variable region domain or light chain variable region domain of anti-PSK antibody of the embodiment (A) and the embodiment (B) may also have one or more deletions, substitutions, insertions, or additions of amino acids. The antigen binding site, which is formed with the heavy chain variable region domain and light chain variable region domain containing polypeptides with deletions, substitutions, insertions, or additions, binds to the same epitope to which 2G9 antibody or 5G5 binds. The cytotoxic activity of PSK can be suppressed by binding of the antibody.

More specifically, there are preferably three or less, more preferably two or less, and most preferably one deletions, substitutions, insertions, or additions of amino acids in each polypeptide. Further, although not specifically limited, for amino acid substitution, it is preferable that a hydrophilic amino acid is substituted with a hydrophilic amino acid, a hydrophobic amino acid is substituted with a hydrophobic amino acid, a basic amino acid is substituted with a basic amino acid, and an acidic amino acid is substituted with an acidic amino acid. When an amino acid is substituted with an amino acid having similar properties, the conformational structure of a protein can be well maintained and the conformational structure of the antigen binding site in the anti-PSK antibody is also maintained, and therefore the anti-PSK antibody can bind to PSK.

When leucine, lysine, and histidine as a cationic amino acid are substituted for each other, asparaginic acid and glutamic acid as an anionic amino acid are substituted for each other, phenylalanine, tryptophan, and tyrosine as an aromatic hydrophobic amino acid are substituted for each other, valine, leucine, methionine, and isoleucine as a hydrophobic amino acid is substituted for each other, and serine and threonine as an amino acid containing hydroxyl group are substituted for each other, the conformational structure of a protein can be maintained well, and thus the binding of antigen-binding site in the anti-PSK antibody can be also maintained.

### Embodiment (C)

As a third embodiment of an anti-PSK antibody (herein after referred to as the embodiment (C)), an antibody competing with the anti-PSK antibody (e.g., the 2G9 antibody) of the embodiment (A) for binding to an epitope, specifically, an antibody which binds to the same epitope as the epitope of PSK to which the anti-PSK antibody (e.g., the 2G9 antibody) of the embodiment (A) binds, may be mentioned.

It is highly likely that the epitope of PSK to which the anti-PSK antibody of the embodiment (A) binds is an epitope present at the physiologically active site of PSK for exhibiting the cytotoxic activity or an epitope near that site. The binding of the anti-PSK antibody of the embodiment (A) to the epitope can suppress the activity of physiologically active site of PSK showing the cytotoxic activity. Further, it is highly likely that the epitope of PSK to which the anti-PSK antibody of the embodiment (A) binds is an epitope present at the physiologically active site of PSK for exhibiting the TGF-β1 inhibiting activity or an epitope near the that site. The binding of the anti-PSK antibody of the embodiment (A) to the epitope can suppress the activity of physiologically active site of PSK showing the TGF-β1 inhibiting activity.

### Embodiment (D)

As a fourth embodiment of an anti-PSK antibody (hereinafter referred to as the embodiment (D)), an antibody competing with the anti-PSK antibody (e.g., 5G5 antibody) of the embodiment (B) for binding to an epitope, specifically, an antibody which binds to the same epitope as the epitope of PSK to which the anti-PSK antibody (e.g., 5G5 antibody) of the embodiment (B) binds, may be mentioned.

It is highly likely that the epitope of PSK to which the anti-PSK antibody of the embodiment (B) binds is an epitope present at the physiologically active site of PSK for exhibiting the cytotoxic activity or an epitope near that site. The binding of the anti-PSK antibody of the embodiment (B) to the epitope can suppress the activity of physiologically active site of PSK showing the cytotoxic activity. Further, it is highly likely that the epitope of PSK to which the anti-PSK antibody of the embodiment (B) binds is an epitope present at the physiologically active site of PSK for exhibiting the TGF-β1 inhibiting activity or an epitope near that site. The binding of the anti-PSK antibody of the embodiment (B) to the epitope can suppress the activity of physiologically active site of PSK showing the TGF-β1 inhibiting activity.

As used herein, the term “antibody which competes for binding to an epitope” includes all antibodies showing competitiveness in an epitope competition test using the two antibodies to be tested. The competition ratio can be calculated from an epitope competition test using two antibodies to be tested. The “antibody which competes for binding to an epitope” may exhibit competition ratio of between 1% and 100%, and specifically, it includes an antibody which exhibits competition ratio of 10% or more, 20% or more, 30% or more, 40% or more, 50% or more, 60% or more, 70% or more, 80% or more, or 90% or more.

Further, the term “ . . . binds to the same epitope as . . . ” means that the epitopes to which an antigen binding site of an antibody binds are identical, and such antibody shows competitiveness in an epitope competition test using two antibodies. The competition ratio in an epitope competition test using an antibody which “binds to the same epitope” is not specifically limited, because the competition ratio in an epitope competition test is determined by titre, binding constant, dissociation constant, affinity constant, and the like of the two antibodies. Accordingly, the antibody which “binds to the same epitope” may exhibit competition ratio of between 1% and 100%. Specifically, the “antibody which competes for binding to an epitope” includes an antibody which exhibits competition ratio of 10% or more, 20% or more, 30% or more, 40% or more, 50% or more, 60% or more, 70% or more, 80% or more, or 90% or more.

The epitope competition test can be carried out by the following method. PSK is coated overnight at 4° C. on a 96 well plate at a concentration of 1 μg/well. After blocking with 1% BSA, a PSK-immobilized plate is prepared. Then, for example, the first antibody is added at concentration of 0.1 μg/mL, 0.5 μg/mL, 1 μg/mL, or 5 μg/mL and incubated at 25° C. for 3 hours. After washing each well with TBST three times, the second antibody solution conjugated with HRP, which has been prepared to have concentration of 0.5 μg/mL, is added thereto and incubated for 1 hour at 25° C. After washing each well with TBST three times, ABST is added as a substrate and a chromogenic reaction is allowed occur for 15 min. After terminating the chromogenic reaction by using Peroxidase Stop Solution, absorbance at 405 nm is measured using a plate reader, and then competition ratio is calculated.

### (Additional Remarks)

The anti-PSK antibody may be a polyclonal antibody, a monospecific antibody, and a monoclonal antibody. Preferably, it is a monoclonal antibody. Further, the animal species for preparing the anti-PSK antibody is not limited, and examples thereof include mammals (such as a mouse, a rat, a rabbit, a human, a sheep, a goat, a cow, a horse, a camel, a pig, a dog, or a cat), amphibians (such as an African clawed frog (Xenopus laevis)), birds (such as a chicken), cartilaginous fishes, and teleost fishes.

The mammalian anti-PSK antibody is classified into five isotypes (IgG, IgA, IgM, IgD, and IgE) depending on the class of H chain as described above. As long as the characteristics of the anti-PSK antibody of the present invention are preserved, the isotypes thereof are not specifically limited. However, IgG or IgM is preferable, and IgM is most preferable. This is because the site responsible for inducing cytotoxic activity related to the anti-tumor activity of PSK, a site showing a binding activity for TGF-β1, and a site responsible for inducing cytokine production can be sufficiently suppressed when the molecular weight of immunoglobulin is large.

Further, the anti-PSK antibody may be a diabody, a single chain molecule, and a multi specific antibody consisting of antibody fragments. The single chain antibody molecule is a single chain Fv (scFv) wherein Fv of a heavy chain and Fv of a light chain are linked to each other. The diabody is a small antibody fragment having two antigen binding sites, wherein a heavy chain variable region domain (V_{H}) is linked to a light chain variable region domain (V_{L}) in a polypeptide chain having the same fragments (V_{H}-V_{L}). Further, a labeled antibody in which various labels are bound to an antibody by a known method, an antibody fused with other material (for example, polypeptide), and an immunotoxin are also included in the anti-PSK antibody of the present invention.

The affinity constant of the anti-PSK antibody is not specifically limited. However, preferably the affinity constant is at least 10⁵ to 10⁹ M⁻¹. Most preferably, the affinity constant is 10⁵M⁻¹ or more. The binding affinity can be measured by Scatchard assay described in Munson et al., Anal. Biochem. 107: 220 (1980).

### (Method for Producing Anti-PSK Antibody)

The anti-PSK antibody can be prepared according to a method well known in the field with PSK is used as an immunogenic antigen. For example, a monoclonal antibody can be prepared according to the method described by Koehler and Milstein (Nature 256: 495-497, 1975). The immunogenic antigen for obtaining the anti-PSK antibody is not specifically limited, as long as PSK has anti-tumor activity. For example, PSK obtained by extracting cell bodies of Coriolus versicolor (Fr.) Quel. Strain CM101 [FERM-P2412 (ATCC20547)] with an aqueous solution such as hot water or alkali solution (for example, hydroxides of alkali metals, in particular, an aqueous solution of sodium hydroxide) followed by purification and drying, can be used.

A hybridoma for preparing the anti-PSK antibody can be obtained from an animal immunized with the antigen described above. For example, a BALB/C mouse is immunized with PSK at regular intervals. After monitoring an increase in antibody titre, PSK dissolved in phosphate buffered physiological saline (PBS) is injected into the tail vein. Two or three days later, the mouse spleen which contains lymphocytes for producing antibodies is aseptically harvested. The lymphocytes can, for example, be established as a monoclonal antibody-producing hybridoma by a method of fusing lymphocytes with myeloma cells in the presence of polyethylene glycol.

When cell fusion is carried out, for example, lymphocytes are fused with myeloma cells in the presence of polyethylene glycol. Various known cells can be used as the myeloma cells for example:SP2/0-Ag14 and P3U1 cells. The fused cells are selected by using a selection medium such as HAT medium which can destroy non-fused cells. Thereafter, presence or absence of a newly produced antibody is screened for culture supernatant of cultured hybridoma. The screening can be carried out by measuring production of PSK specific antibody using an enzyme linked immunosorbent assay (ELISA).

The hybridoma can be subcultured by using any known medium, for example, RPMI1640. The monoclonal antibody can be prepared by culturing the obtained hybridoma, for example, after adding 10% bovine serum to RPMI1640 and culturing the hybridoma at 37° C. in the presence of 5% CO₂, antibodies are accumulated in a culture supernatant. Further, by intraperitoneal injection of the hybridoma to a mouse and collecting ascites therefrom, it is possible to produce the antibody in ascites. The monoclonal antibody can be purified according to a method well known in the art. For example, it can be purified by a method of using an affinity column to which PSK is bound, a method of purification using an ion exchange column, a method of purification using Protein G, or a combined method thereof.

The anti-PSK antibody can be also constructed by genetic engineering, i.e., by linking a DNA which encodes the polypeptides of the heavy chain variable region domain and light chain variable region domain of the 2G9 antibody, to the DNA of a constant region in a heavy chain and light chain of immunoglobulin. Further, the anti-PSK antibody can be also used effectively for the method for analyzing PSK and a kit for analyzing PSK, as described below.

### (Method for Producing Chimeric Antibody and CDR-Grafted Antibody)

A chimeric antibody can be constructed by linking the heavy chain variable region domain and light chain variable region domain of an anti-PSK antibody to a polypeptide of a constant region in an antibody of mammals other than human. Further, it can be also prepared by linking the heavy chain variable region domain and light chain variable region domain of an anti-PSK antibody to a polypeptide of a constant region in IgW, IgNAR, IgX, or IgY. The CDR-grafted antibody can be constructed by linking three heavy chain variable region domains and three light chain variable region domains of an anti-PSK antibody to a framework region of an antibody of mammals other than human. Further, it can be also prepared by linking the heavy chain variable region domain and light chain variable region domain of an anti-PSK antibody to a polypeptide of a framework region in IgW, IgNAR, IgX, or IgY.

### (Antigen Binding Fragment)

The antigen binding fragment of the invention means Fab, Fab′, F(ab′)₂, and Fv fragments of each anti-PSK antibody described above. These antigen binding fragments can be obtained by, for example, digesting the antibody with a proteolytic enzyme (e.g., pepsin, papain, or the like) according to a standard method and purifying it by a known method for purifying a protein. As used herein, the term “antigen binding fragment” means a fragment of an antibody which is capable of binding to an epitope of PSK. Further, the diabody, single chain antibody molecule, and multi specific antibody prepared using antibody fragments, which are produced by a genetic engineering method, may be also categorized as an antigen binding fragment.

### Embodiment 2

### [2] Method for Analyzing PSK

The method for analyzing PSK and kit for analyzing PSK are explained herein below as embodiment 2. The terms used in this embodiment have the same meanings as those described in the embodiment 1, unless specifically described otherwise. First, the method for analyzing PSK is explained below.

The method for analyzing PSK according to the embodiment is an immunological analysis which is characterized in that the anti-PSK antibody or an antigen binding fragment of the antibody described in embodiment 1 is used. Specifically, by using at least one of a polyclonal antibody, a monospecific antibody, or a monoclonal antibody against PSK, or a chimeric antibody, a CDR-grafted antibody, or a human type antibody thereof, or a Fab, Fab′, F(ab′)₂, Fv fragment of the antibody, a diabody, a single chain antibody molecule, or a multi specific antibody, the method for analyzing PSK according to the embodiment can be carried out. Physiologically active PSK can be analyzed by using the method for analyzing PSK according to the present embodiment. The method for analyzing PSK is not specifically limited, as long as PSK can be quantitatively or semi-quantitatively determined or the presence or absence of PSK can be determined, by using the anti-PSK antibody. Possible methods include: an enzyme immunoassay, an immunohistochemical staining, a surface Plasmon resonance (SPR method: Biacore method), a latex agglutination immunoassay, a chemiluminescence assay, a fluorescent antibody method, a radioimmunoassay, an immuno precipitation, and a Western blotting.

The term “analyzing” is used herein to mean both “measuring” for determining quantitatively or semi-quantitatively an amount of an analyte and “detecting” the presence or absence of an analyte.

When an enzyme immunoassay, e.g., ELISA, is used as an analysis method, PSK can be detected with high accuracy and quantitative performance by using the anti-PSK antibody described in the embodiment 1 as a capturing antibody and a detecting antibody.

Specifically, it includes steps of: immobilizing a capturing antibody by which an antibody is immobilized on a surface of a reaction system, supplying an antigen by which a sample to be tested is supplied to the reaction system, supplying a detecting antibody by which an antibody labeled by a detecting enzyme is supplied to the reaction system, supplying a chromogenic substrate by which a chromogenic substrate for the detecting enzyme is supplied to the reaction system, and detecting the chromogenic reaction by which reaction between the detecting enzyme and the chromogenic substrate is detected.

Concrete procedures of sandwich ELISA will now be described. First, on an insoluble carrier such as a micro plate or beads, an antibody capable of binding to PSK is immobilized (i.e., a capturing antibody or a primary antibody). Then, to prevent non-specific adsorption on the capturing antibody or the insoluble carrier, the insoluble carrier is blocked with an appropriate blocking agent (e.g., bovine serum albumin or gelatin). A test sample which may contain PSK and a primary reaction solution are added to the insoluble carrier (plate or beads) on which the capturing antibody is immobilized, so that the capturing antibody is brought into contact with PSK for binding thereto (primary reaction process). Thereafter, antigens that are not bound with the capturing antibody and impurities are washed with an appropriate washing solution (for example, a phosphate buffer solution containing a surface active agent). Then, a labeled antibody (i.e., secondary antibody), wherein a antibody capable of binding to the captured PSK and an enzyme such as horse radish peroxidase (HRP) are conjugated, are added so that the captured antigen is linked with the labeled antibody (secondary reaction process). As result of this reaction, an immune complex of capturing-antibody-PSK-labeled antibody is formed on a carrier such as a micro plate. The unbound labeled antibody is washed with a washing solution. A chromogenic substrate or a light-emitting substrate for the enzyme linked to the labeled antibody is added to allow the reaction between the enzyme and the substrate, and the signal is detected.

Further, using the sandwich ELISA, one kind of an antibody (e.g., 2G9 antibody) may be used as a capturing antibody (primary antibody) and also as a labeled antibody (secondary antibody). In other words, when an antibody can bind to multiple identical epitopes present on a single PSK molecule, sandwich ELISA can be carried out using only one kind of such antibody.

When immunohistochemical staining is used as an analytical method, immunohistochemical staining can be carried out according to a known immunohistochemical staining method—using the anti-PSK antibody. For example, a tissue specimen obtained from a patient administered with PSK is prepared according to a standard method. Then a biotinylated anti-PSK antibody is added to the specimen. Next, streptavidin conjugated with HRP is added, and the chromogenic reaction is carried out by adding DAB substrate (manufactured by DAKO).

Alternatively, after obtaining a tissue specimen bound with an anti-PSK antibody, anti-mouse IgM antibody labeled with HRP is added thereto so as to bind to the anti-PSK antibody as a secondary antibody. Staining is then carried out by a treating with 3,3′-diaminobenzidine. After the staining, microscopic observation is performed. The area stained with brown color is found as an area in which PSK is expressed.

When a surface Plasmon resonance method is used as a method for analysis, it can be carried out according to a known surface Plasmon resonance method using the anti-PSK antibody. Concretely, by using a Surface Plasmon Resonance sensor (SPR sensor), an anti-PSK antibody is immobilized on the surface of a sensor chip. A test sample which may contain PSK is then brought into contact with the sensor chip to allow an antigen-antibody reaction. Then, a subtle change in the metal surface caused by binding between an antigen and an antibody is detected by utilizing an optical phenomenon, i.e., surface Plasmon resonance, and visualized by a sensorgram. Since surface Plasmon resonance is a method for direct measurement of an optical change, it is not necessary to label the anti-PSK antibody. Further, it can be carried out within a short period of time, and detection can be made even with a small amount of a test sample. A Biacore 3000 (trade name, manufactured by Biacore) can be used as the apparatus for measurement. A CM5 chip added with a carboxymethyl group can be used as the sensor chip.

Examples of the test sample that can be analyzed using—the method for analyzing PSK include, PSK, in particular: a drug, a food or a drink which may contain physiologically active PSK, or a sample of a living body or a sample derived from a living body of a patient administered with PSK. Specific examples of the drug, food, or drink include a pharmaceutical composition or a pharmaceutical preparation, or a hot water and/or alkali extract originating from a microorganism, which is used as a starting material of a pharmaceutical product, a health product or a functional food product, or a hot water and/or alkali extract originating from a microorganism, which is used as a starting material of a health or functional food product. Further, examples of the sample of a living body or the sample derived from a living body include: urine, blood, serum, plasma, feces, bone marrow fluid, saliva, cells, tissues, or organs, or preparations thereof (e.g., biopsy sample).

By using the method for analyzing PSK, PSK obtained by certain hot water and/or alkali extraction can be qualitatively and quantitatively detected from blood or tissue after administering pharmaceutical products or foods and PSK. Thus, the intake amount (administration amount) of physiologically active PSK can be calculated, which is very useful. For example, as the blood concentration of PSK after PSK administration and level of PSK delivered to a tumor can be determined conveniently and highly accurately, in vivo kinetics or efficacy can be also evaluated conveniently and highly accurately.

### [3] Kit for Analyzing PSK

The kit for analyzing PSK according to the embodiment is an analysis kit which is characterized in that the anti-PSK antibody or an antigen binding fragment of the antibody described in the embodiment 1, is included. The kit for analyzing PSK can be particularly useful for analyzing physiologically active PSK. Further, the kit for analyzing PSK may be a kit which is used for: an enzyme immunoassay, an immunohistochemical staining, a surface Plasmon resonance (SPR method: Biacore method), a latex agglutination immunoassay, a chemiluminescence assay, a fluorescent antibody method, a radioimmunoassay, an immuno precipitation, or a Western blotting.

When the kit for analyzing PSK is a kit used for enzyme immunoassay (for example, ELISA), it has a combination appropriately including: a carrier (e.g., micro plate, microtube, or paper) whose surface is immobilized with an anti-PSK antibody as a capturing antibody, an anti-PSK antibody labeled with a detecting enzyme as a detecting antibody (i.e., labeled antibody), a detecting enzyme, a chromogenic substrate therefor, and other reagents for ELISA (e.g., washing solution).

When the kit for analyzing PSK is a kit used for immunohistochemical staining, it may include: a biotinlyated anti-PSK antibody of the invention, HRP-conjugated streptavidin, DAB substrate, or non-labeled anti-PSK antibody, HRP-conjugated anti-mouse IgG antibody, a substrate, or the like.

When the kit for analyzing PSK is a kit used for SPR analysis, it may include a sensor chip immobilized with the anti-PSK antibody of the present invention or the like.

Thus, the kit for analyzing PSK may contain an anti-PSK antibody or a fragment thereof in appropriate form according to the immunological method to be used. Specific examples of the labeling substance include peroxidase, alkali phosphatase, β-D-galactosidase, or glucosidase as an enzyme; fluorescein isocyanate or rare earth metal chelate as a fluorescent substance; ³H, ¹⁴C, and ¹²⁵I as a radioisotope; and also biotin, avidin, or a chemiluminescence substance. In addition, in the case of an enzyme or a chemiluminescent substance, since they cannot develop a measurable signal by themselves, a substance corresponding to each enzyme or chemiluminescent substance is preferably contained therein.

The kit for analyzing PSK of the present invention can analyse PSK with physiological effects, and it may contain instruction describing how. It is also possible to describe on a package of a kit that PSK with physiological effects can be analyzed by using the kit.

The invention is not limited by each embodiment described above, and within the scope of the claims, various modifications can be made. Further, embodiments that are obtained by appropriate combination of technical means described in different embodiments are also within the scope of the invention.

### EXAMPLES

The invention will be explained in greater detail in view of the Examples, but the scope of the invention is not limited by them.

### Example 1

### Production of Antibody Against PSK

Antibody production was performed in the order: (1) immunization with an antigen, (2) measurement of antibody titre in anti-serum, and (3) production of anti-PSK monoclonal antibody. Details of each step:

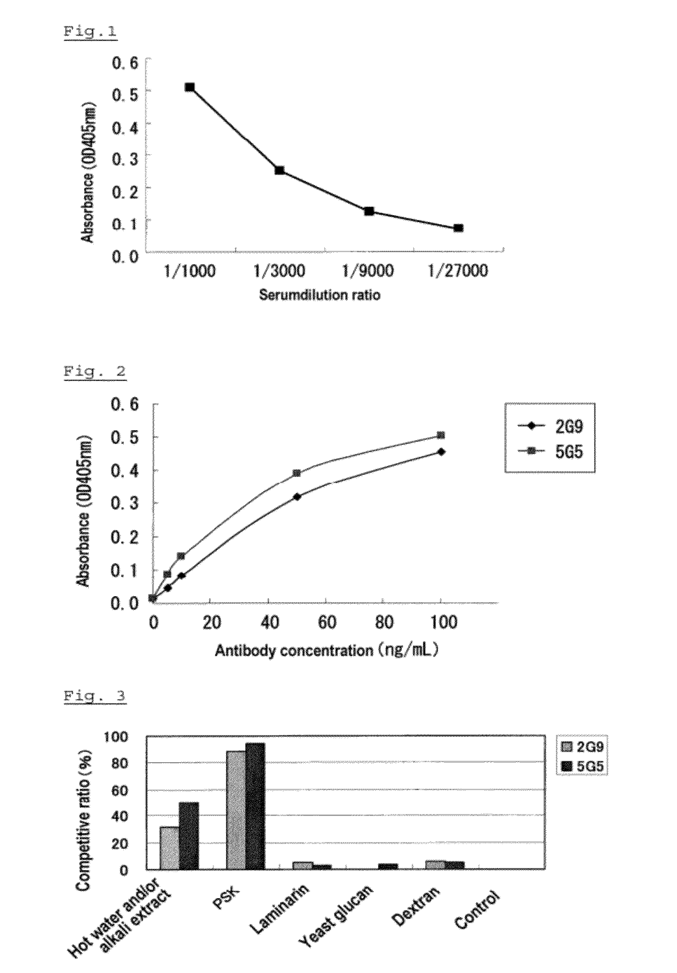

(1) Immunization with antigen: For the first immunization, an equal amount of phosphate buffered saline (hereinafter referred to as “PBS”) solution of PSK and Freund's Complete Adjuvant (manufactured by Sigma-Aldrich Company) were mixed, and by using an ultrasonicator, a highly viscous emulsion was prepared. The emulsion was subcutaneously injected to a 6-week old female Balb/c mouse (provided by Oriental Yeast Company) such that PSK is administered in an amount of 0.1 mg/animal. After one week, a second immunization was carried out. Specifically, PBS solution of PSK and Freund's Incomplete Adjuvant (manufactured by Sigma-Aldrich Company) were mixed to give an emulsion and the emulsion was intraperitoneally injected to a mouse such that PSK is administered in an amount of 0.1 mg/animal. The immunization was carried out every week with the same procedure. After the eighth immunization, blood was taken from the tail vein and antibody titre was measured. Boosting was performed by intraperitoneal injection of PSK to the animal in which the antibody titer to PSK was raised. Thereafter, cell fusion was carried out obtaining hybridoma.
(2) Measurement of antibody titre in anti-serum: After the eighth immunization, the antibody titre in each serum (i.e., anti-serum) obtained from the Balb/c mouse was measured by ELISA. The specific procedures are as follows. On a 96 well plate, the PSK solution was added to give 1 μg/well and allowed to react overnight at 4° C. to immobilize the PSK. After blocking with 1% BSA, ×1,000 diluted solution of the obtained serum was added to each well in an amount of 50 μL and allowed to react for 3 hours at 25° C. Then, each well was washed three times with TBS with 0.05% Tween 20 added (hereinafter referred to as “TBST”). 50 μL of a mouse IgM antibody solution which is conjugated with HRP and prepared to have a concentration of 1 μg/mL was added to each well and allowed to react for 1 hour at 25° C. After washing each well three times with TBST, ABST (manufactured by KPL Company) was added to allow color development for 15 minutes. The color development reaction was terminated by adding 50 μL of Peroxidase Stop Solution (manufactured by KPL Company), and the absorbance at 405 nm was measured by means of a plate reader. FIG. 1 illustrates a result of antibody titre measured by ELISA, wherein the horizontal axis of the graph indicates serum dilution ratio and the vertical axis of the graph indicates absorbance (titre).
(3) Production of anti-PSK monoclonal antibody: Using the animal, in which the antibody titre to PSK was raised by PSK immunization, a monoclonal antibody was prepared by following a standard method. Specifically, seven days after the boosting, the mouse spleen was collected and the spleen cells were fused with mouse myeloma cell line P3U1. By culturing the cells for 2 to 3 weeks in a HAT selection medium, a hybridoma colony was obtained. Culture supernatant was collected from each well and hybridoma screening was performed according to the ELISA method described in (2) above. The same screening was repeated twice for the positive hybridomas which produce PSK antibody, to select the hybridomas having excellent antibody producing ability or proliferation property. As a result, from about 100 positive hybridomas, two hybridomas one producing 2G9 antibody and one producing 5G5 antibody were selected. The 2G9 antibody and 5G5 antibody were both found to be an IgM antibodies.

Mass preparation of each antibody was performed by using mouse ascites. Specifically, 500 μL of pristan was intraperitoneally administered to a female Balb/c mouse. Seven to ten days later, each animal received about 10⁷ hybridomas. One to two weeks later, when ascites has accumulated, it is collected time to time and stored at −80° C. until purification. Purification of the antibody from the ascites was performed as follows. The ascites collected was added to phosphate buffer (pH 7.5) to a final concentration of 25 mM, and filtered through a 0.45 μm filter. The resultant was applied to a Protein G column and the flow through fraction was collected. Then, according to a standard method, the IgM fraction was collected by using a HiTrap IgM column (trade name, manufactured by Amersham) or a Sepharose HP column (trade name, manufactured by Amersham). Further, the obtained IgM fraction was further fractionated using Sepharose 200 pg column to purify pentamer IgM. The titre of 2G9 antibody and 5G5 antibody is shown in FIG. 2.

### Example 2

### Determination of Specificity of 2G9 Antibody and 5G5 Antibody

In order to investigate the specificity of 2G9 antibody and 5G5 antibody, competition ELISA was performed by using laminarin, yeast glucan, and dextran as polysaccharides, and also PSK and hot water and/or alkali extract of Coriolus versicolor (Fr.) Quel. The laminarin, yeast glucan, and dextran used were purchased from Sigma Company.

A 96 well plate was coated overnight at 4° C. with PSK to give a concentration of 1 μg/well. After blocking with 1% BSA, a PSK-immobilized plate was prepared. 2G9 antibody or 5G5 antibody of 0.5 μg/mL concentration, and 5 μg/mL of laminarin, yeast glucan, or dextran were reacted at 37° C. for 3 hours. The reaction solution in which the antibody and polysaccharides are reacted was added to each well of the immobilized plate and incubated at 25° C. for 3 hours. Then, each well was washed three times with TBST, and 50 μL of a solution of a mouse IgM antibody labeled with HRP (concentration of 1 μg/mL) was added to each well and incubated for 1 hour at 25° C. After washing each well three times with TBST, as a substrate, ABST was added to allow color development for 15 minutes. The color development reaction was terminated by adding 50 μL of Peroxidase Stop Solution, and the absorbance at 405 nm was measured by using a plate reader. As a result, as shown in FIG. 3, the reactivity of 2G9 antibody and 5G5 antibody is inhibited by PSK and hot water and/or alkali extract of Coriolus versicolor (Fr.) Quel., but not by laminarin, yeast glucan, or dextran. Thus, it was found that the 2G9 antibody and the 5G5 antibody recognize an epitope which is present in PSK but not in laminarin, yeast glucan, or dextran.

Further, competition ELISA was performed by using protein-hydrolyzed PSK wherein protein parts of the PSK are hydrolyzed by treating PSK with hydrazine. Specifically, by adding 2 mL of anhydrous hydrazine to 10 mg of vacuum dried PSK and treating for 12 hours at 100° C., protein-hydrolyzed PSK was obtained. 5 μg/mL of the protein-hydrolyzed PSK was reacted for three hours at 37° C. with 0.5 μg/mL 2G9 antibody or 5G5 antibody, and then the competition ELISA was carried out according to the method described above. As a result, it was found that the reactivity of 2G9 antibody and 5G5 antibody to PSK is lowered by protein-hydrolyzed PSK (FIG. 4). Thus, it is believed that the 2G9 antibody and 5G5 antibody recognize protein-hydrolyzed PSK wherein the protein parts of the PSK are hydrolyzed.

### Example 3

### Epitope Competition Test Using 2G9 Antibody and 5G5 Antibody

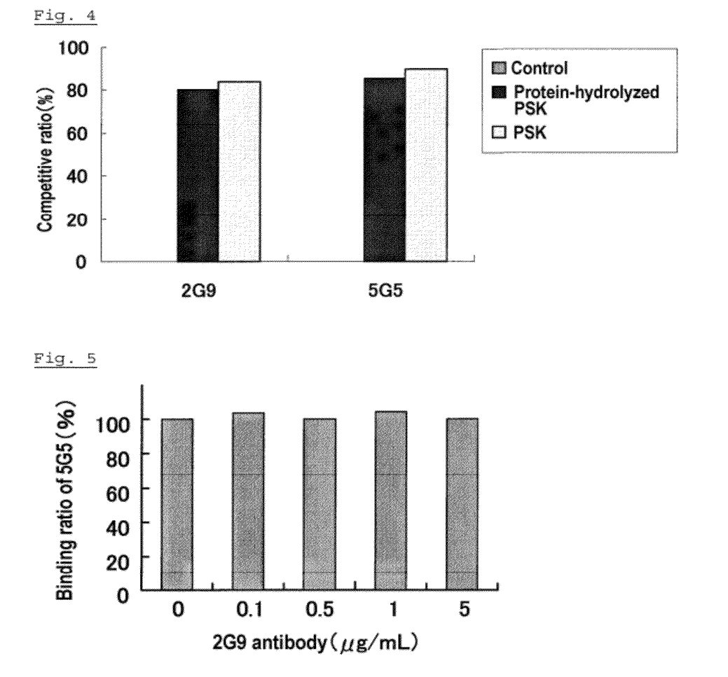

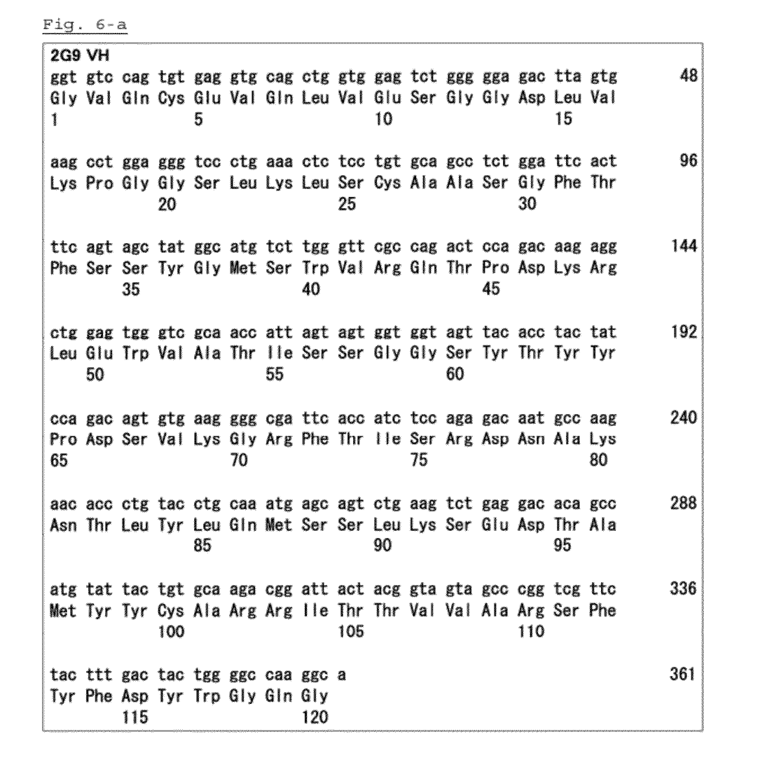

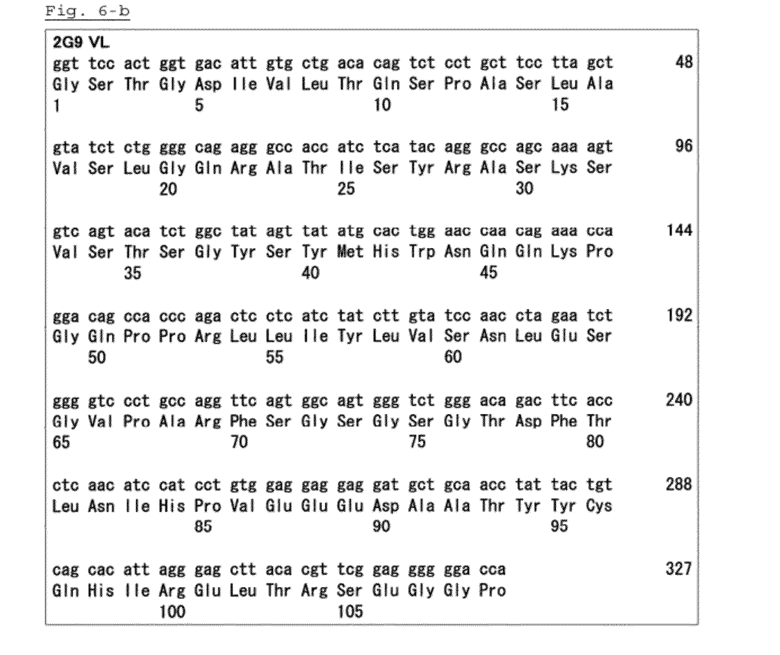

Epitope competition test was performed using 2G9 antibody and 5G5 antibody. Specifically, a 96 well plate was coated overnight at 4° C. with PSK to give a concentration of 1 μg/well. After blocking with 1% BSA, a PSK-immobilized plate was prepared. 2G9 antibody of 0.1, 0.5, 1, or 5 μg/mL concentration was added and incubated at 25° C. for 3 hours. Then, each well was washed three times with TBST, and solution of 5G5 antibody conjugated with HRP (concentration of 0.5 μg/mL) was added to each well and incubated for 1 hour at 25° C. After washing each well three times with TBST, as a substrate, ABST was added to allow color development for 15 minutes. The color development reaction was terminated by adding Peroxidase Stop Solution, and the absorbance at 405 nm was measured by using a plate reader. As a result, as shown in FIG. 5, binding of 5G5 antibody was not inhibited by 2G9 antibody, and therefore it was found that the epitope of 2G9 antibody and the epitope of 5G5 antibody are not close to each other.

### Example 4

### Determination of Sequence of Variable Region in 2G9 Antibody and 5G5 Antibody

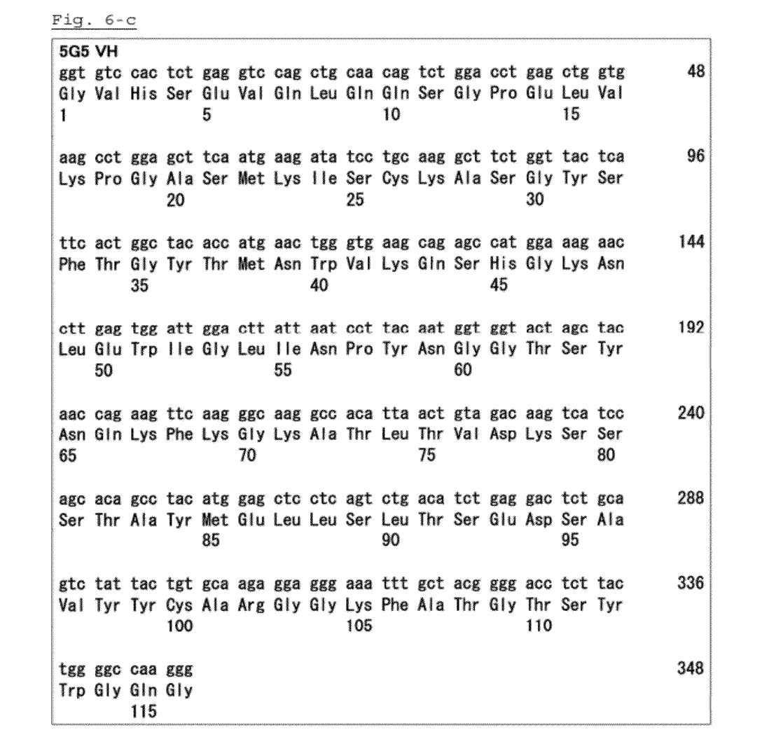

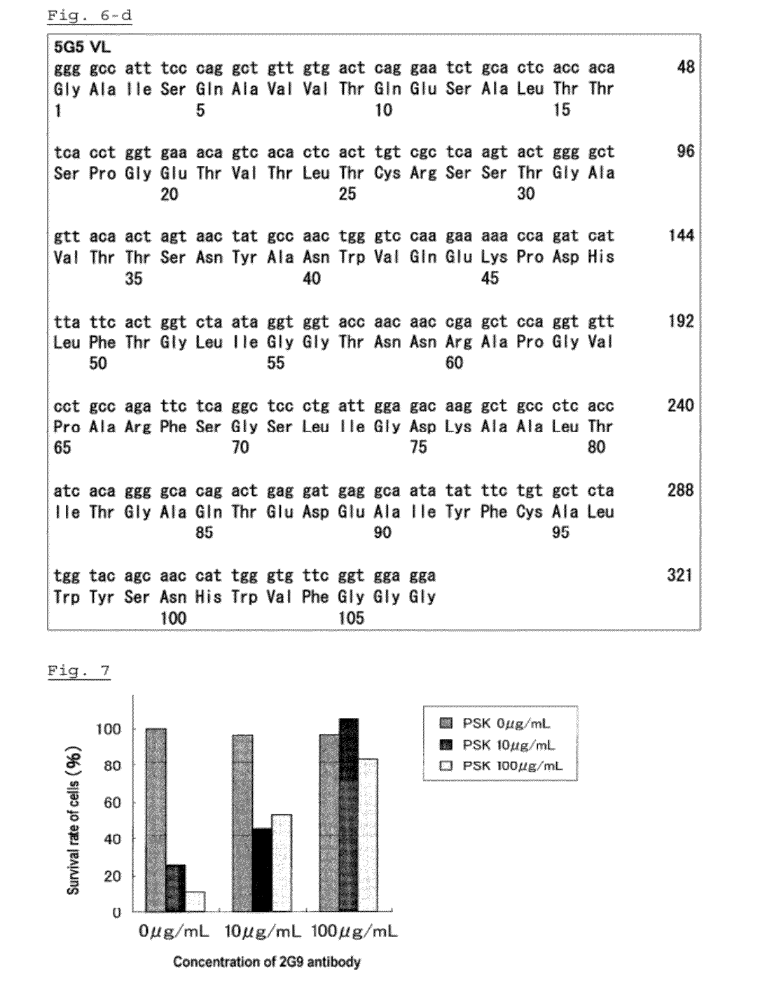

From the hybridoma which produces 2G9 antibody or 5G5 antibody, total RNAs were extracted by a standard method, and by carrying out a reverse transcription reaction using an oligo dT primer, cDNA was prepared. To amplify the gene of the variable region from the cDNA obtained, PCR was performed using a mouse Ig primer set (Novagen) according to the instructions included therein. The resulting gene of the antibody variable region was cloned into TA cloning vector, i.e. pCR2.1 vector, and subjected to sequencing. Base sequences of the nucleotides of the heavy chain variable region domain and light chain variable region domain in 2G9 antibody and base sequences of the nucleotides of the heavy chain variable region domain and light chain variable region domain in 5G5 antibody are given in FIG. 6. Further, the amino acid sequence of H-FR1, H-CDR1, H-FR2, H-CDR2, H-FR3, H-CDR3, H-FR4, L-FR1, L-CDR1, L-FR2, L-CDR2, L-FR3, L-CDR3, and L-FR4 of each antibody is given below.

Amino acid sequence of the heavy chain variable
region domain of 2G9 antibody:
(SEQ ID NO: 4)
H-FR1: GVQCEVQLVESGGDLVKPGGSLKLSCAASGFTFS
(SEQ ID NO: 6)
H-CDR1: SYGMS
(SEQ ID NO: 8)
H-FR2: WVRQTPDKRLEWVA
(SEQ ID NO: 10)
H-CDR2: TISSGGSYTYYPDSVKG
(SEQ ID NO: 12)
H-FR3: RFTISRDNAKNTLYLQMSSLKSEDTAMYYCAR
(SEQ ID NO: 14)
H-CDR3: RITTVVARSFYFDY
(SEQ ID NO: 16)
H-FR4: WGQG
Amino acid sequence of the light chain variable
region domain of 2G9 antibody:
(SEQ ID NO: 20)
L-FR1: GSTGDIVLTQSPASLAVSLGQRATISY
(SEQ ID NO: 22)
L-CDR1: RASKSVSTSGYSYMH
(SEQ ID NO: 24)
L-FR2: WNQQKPGQPPRLLIY
(SEQ ID NO: 26)
L-CDR2: LVSNLES
(SEQ ID NO: 28)
L-FR3: GVPARFSGSGSGTDFTLNIHPVEEEDAATYYC
(SEQ ID NO: 30)
L-CDR3: QHIRELTRS
(SEQ ID NO: 32)
L-FR4: EGGP
Amino acid sequence of the heavy chain variable
region domain of 5G5 antibody:
(SEQ ID NO: 36)
H-FR1: GVHSEVQLQQSGPELVKPGASMKISCKASGYSFT
(SEQ ID NO: 38)
H-CDR1: GYTMN
(SEQ ID NO: 40)
H-FR2: WVKQSHGKNLEWIG
(SEQ ID NO: 42)
H-CDR2: LINPYNGGTSYNQKFKG
(SEQ ID NO: 44)
H-FR3: KATLTVDKSSSTAYMELLSLTSEDSAVYYCAR
(SEQ ID NO: 46)
H-CDR3: GGKFATGTSY
(SEQ ID NO: 48)
H-FR4: WGQG
Amino acid sequence of the light chain variable
region domain of 5G5 antibody:
(SEQ ID NO: 52)
L-FR1: GAISQAVVTQESALTTSPGETVTLTC
(SEQ ID NO: 54)
L-CDR1: RSSTGAVTTSNYAN
(SEQ ID NO: 56)
L-FR2: WVQEKPDHLFTGLIG
(SEQ ID NO: 58)
L-CDR2: GTNNRAP
(SEQ ID NO: 60)
L-FR3: GVPARFSGSLIGDKAALTITGAQTEDEAIYFC
(SEQ ID NO: 62)
L-CDR3: ALWYSNHWV
(SEQ ID NO: 64)
L-FR4: FGGG

### Example 5

### Neutralization Effect Against Cytotoxic Activity of PSK

PSK has an activity of directly damaging tumor cells. In this example, the effect of neutralizing cytotoxic activity of PSK by 2G9 antibody and 5G5 antibody was determined. First, PSK sensitive cancer cell line Colon26 (1×10³/well) was cultured overnight on a 96 well plate and PSK (0, 10, or 100 μg/mL) and 2G9 antibody or 5G5 antibody (0, 10, or 100 μg/mL) were added followed by further culture for three days. The number of the cells after culturing was estimated by MTT assay. As a result, it was found that the proliferation of Colon26 cells was inhibited by PSK in a concentration dependent manner. However, the proliferation was recovered by addition of 2G9 antibody and 5G5 antibody also in a concentration dependent manner. These results suggest that 2G9 antibody and 5G5 antibody have an activity of suppressing the physiological activity of PSK (i.e., cytotoxic activity). In FIG. 7, the results obtained from 2G9 antibody are shown.

### Example 6

### Immunohistochemical Staining Using Tumor Tissue After Oral Administration of PSK

2G9 antibody and 5G5 antibody prepared in the Example 1 were labeled with biotin. The biotin labeling was carried out by using a Sulfo-OSu Biotinylation Kit (Dojin Chemical Laboratories) according to the protocols attached thereto. Specifically, the antibody solution obtained from the Example 1 was added to a sample tube and sodium hydrogen carbonate buffer solution was added to give a salt concentration of 50 mM and a protein concentration of 5.0 mg/0.5 mL. The mixture was mixed well using a vortex mixer. Thereafter, Biotin-(AC5) 2Sulfo-Osu was prepared to 10 mg/750 μL, and 17.5 μL of the solution was added to the antibody solution. The solutions were mixed well by using a vortex mixer and reacted for 2 hours at 25° C. Subsequently, the reaction solution was purified by gel filtration column to collect the biotinylated antibody solution.

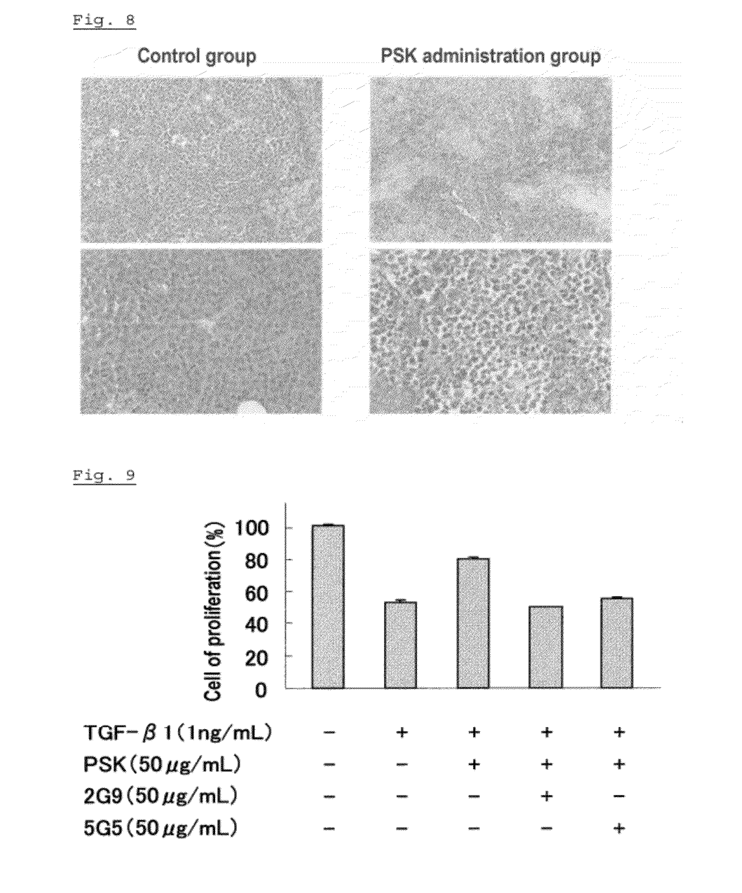

MethA cells (1×10⁶ cells) were subcutaneously implanted to a 6-week old female Balb/c as a tumor cell. A month later, PSK was orally administered to the animal (1000 mg/kg, three times per week). Physiological saline was administered to a control group. Twenty-four hours later, the tumor tissues were harvested and formalin-fixed specimens were prepared according to a standard method. Then, by using the biotinylated 2G9 antibody or 5G5 antibody, immunohistochemical staining was performed according to a standard method. Specifically, 400 μL of 1 μg/mL 2G9 antibody or 5G5 antibody was added to each specimen and incubated for 1 hour at room temperature. The specimen was washed with TBS, 0.1 μg/mL streptavidin HRP was added, and incubated for 1 hour at room temperature. The specimen was washed with TBS, added to DAB substrate (manufactured by DAKO) for color development, and nucleus staining was carried out by using hematoxylin. As a result, it was found that both the 2G9 antibody and 5G5 antibody are stained in the tumor tissues, indicating that PSK is accumulated in the tumor tissues. In FIG. 8, the microscopic image of immunohistochemical staining using 2G9 antibody is shown.

### Example 7

### Suppression of TGF-β1 Inhibitory Activity of PSK by Anti-PSK Antibody

It is reported that PSK binds to TGF-β1, which is an immunosuppressive substance, and neutralizes the activity thereof. In this example, it was determined whether or not the TGF-β1 inhibitory activity of PSK is suppressed by 2G9 antibody or 5G5 antibody. Specifically, the determination was made by using TGF-β1 sensitive Mv1 Lu cells proliferation of which is suppressed by TGF-β1.

PSK (50 μg/mL) and anti-PSK antibody (50 μg/mL) were incubated at 37° C. for 3 hours. Then, hTGF-β1 (1 ng/mL) was added thereto and further incubated for 3 hours. The resultant was added to a 96 well plate in which Mv1 Lu cells (3×10³ cells) were cultured. After culturing for three days, the number of the cells was estimated by MTT assay. As a result, it was shown that 2G9 antibody and 5G5 antibody suppressed the TGF-β1 inhibitory activity of PSK (FIG. 9).

### INDUSTRIAL APPLICABILITY

The anti-PSK antibody, method for analyzing PSK, and the kit for analyzing PSK of the invention can be used for analyzing PSK having a physiological activity, and therefore they are useful for analyzing PSK contained in a drug, a food or a drink containing PSK. Accordingly, they can be also used for product management of such a drug, food or drink.

1. An antibody characterized by recognizing PSK and inhibiting the antitumor activity of PSK.

2. The antibody according to claim 1, wherein the inhibition of the antitumor activity is inhibition of the cytotoxic activity of PSK.

3. The antibody according to claim 1, wherein the inhibition of the antitumor activity is suppression of the TGF-β1 inhibitory activity.

4. The antibody according to claim 1, having
(1) a heavy chain variable region domain comprising: the polypeptide of heavy chain complementarity determining region 1 consisting of an amino acid sequence of SEQ ID NO: 6, the polypeptide of heavy chain complementarity determining region 2 consisting of an amino acid sequence of SEQ ID NO: 10, and the polypeptide of heavy chain complementarity determining region 3 consisting of an amino acid sequence of SEQ ID NO: 14, and a light chain variable region domain comprising: the polypeptide of light chain complementarity determining region 1 consisting of an amino acid sequence of SEQ ID NO: 22, the polypeptide of light chain complementarity determining region 2 consisting of an amino acid sequence of SEQ ID NO: 26, and the polypeptide of light chain complementarity determining region 3 consisting of an amino acid sequence of SEQ ID NO: 30; or
(2) a heavy chain variable region domain comprising: the polypeptide of the heavy chain complementarity determining region 1, the polypeptide of the heavy chain complementarity determining region 2, and the polypeptide of the heavy chain complementarity determining region 3, and a light chain variable region domain comprising: the polypeptide of the light chain complementarity determining region 1, the polypeptide of the light chain complementarity determining region 2, and the polypeptide of the light chain complementarity determining region 3, wherein each polypeptide region consists of an amino acid sequence with one or more amino acid deletions, substitutions, insertions, or additions in at least one of the following amino acid sequences: the amino acid sequence of SEQ ID NO: 6, the amino acid sequence of SEQ ID NO: 10, the amino acid sequence of SEQ ID NO: 14, the amino acid sequence of SEQ ID NO: 22, the amino acid sequence of SEQ ID NO: 26, and the amino acid sequence of SEQ ID NO: 30.

5. The antibody according to claim 1, having
(1) a heavy chain variable region domain comprising: the polypeptide of heavy chain complementarity determining region 1 consisting of an amino acid sequence of SEQ ID NO: 38, the polypeptide of heavy chain complementarity determining region 2 consisting of an amino acid sequence of SEQ ID NO: 42, and the polypeptide of heavy chain complementarity determining region 3 consisting of an amino acid sequence of SEQ ID NO: 46, and a light chain variable region domain comprising: the polypeptide of light chain complementarity determining region 1 consisting of an amino acid sequence of SEQ ID NO: 54, the polypeptide of light chain complementarity determining region 2 consisting of an amino acid sequence of SEQ ID NO: 58, and the polypeptide of light chain complementarity determining region 3 consisting of an amino acid sequence of SEQ ID NO: 62; or
(2) a heavy chain variable region domain comprising the polypeptide of the heavy chain complementarity determining region 1, the polypeptide of the heavy chain complementarity determining region 2, and the polypeptide of the heavy chain complementarity determining region 3, and a light chain variable region domain comprising: the polypeptide of the light chain complementarity determining region 1, the polypeptide of the light chain complementarity determining region 2, and the polypeptide of the light chain complementarity determining region 3, wherein each polypeptide region consists of an amino acid sequence with one or more amino acid deletions, substitutions, insertions, or additions in at least one of the following amino acid sequences: the amino acid sequence of SEQ ID NO: 38, the amino acid sequence of SEQ ID NO: 42, the amino acid sequence of SEQ ID NO: 46, the amino acid sequence of SEQ ID NO: 54, the amino acid sequence of SEQ ID NO: 58, and the amino acid sequence of SEQ ID NO: 62.

6. An antibody which competes with the antibody according to claim 4, for binding to an epitope.

7. An antibody which binds to an epitope to which the antibody according to claim 4 binds.

8. The antibody according to claim 1, wherein the antibody is an IgM antibody.

9. The antibody according to claim 1, wherein the antibody is a chimeric antibody, a CDR-grafted antibody, or a human type antibody.

10. The antibody according to claim 9, wherein the chimeric antibody is a chimeric antibody with a human antibody or the CDR-grafted antibody is a CDR-grafted antibody with a human antibody.

11. The antibody according to claim 9, wherein the chimeric antibody is a chimeric antibody with IgW, IgNAR, IgX, or IgY and the CDR-grafted antibody is a CDR-grafted antibody with IgW, IgNAR, IgX, or IgY.

12. An antigen binding fragment selected from a group consisting of Fab, Fab′, F(ab′)₂, Fv fragment, a diabody, a single chain antibody molecule, and a multi-specific antibody, of the antibody according to claim 1.

13. A method for analyzing PSK using the antibody according to claim 1, or an antigen binding fragment thereof.

14. A kit for analysis of PSK comprising the antibody according to claim 1, or an antigen binding fragment thereof.

15. A method of using the antibody according to claim 1 or an antigen binding fragment thereof, for analysis of PSK.

16. A method of using the antibody according to claim 1, or an antigen binding fragment thereof, for manufacture of a kit for analysis of PSK.

17. An antibody which competes with the antibody according to claim 5, for binding to an epitope.

18. An antibody which binds to an epitope to which the antibody according to claim 5 binds.
